(19) United States
(12) Patent Application Publication
(10) Pub. No.: US 2007/0117157 A1
(43) Pub. Date: May 24, 2007

(54) Parathyroid hormone-like polypeptides

(76) Inventor: Jeffrey David Zajac, Templestowe (AU)

Correspondence Address: SHERIDAN ROSS PC, 1560 BROADWAY, SUITE 1200, DENVER, CO, 80202, US

(21) Appl. No.: 10/490,319

(22) PCT Filed: Sep. 15, 2003
(86) PCT No.: PCT/AU03/01201

§ 371 Date: Oct. 20, 2004

(30) Foreign Application Priority Data
Sep. 13, 2002 (AU) 2002951372

(51) Int. Cl. G01N 33/567; C07H 21/04; A61K 38/29; C07K 14/635

(52) U.S. Cl. 435/7.2; 514/12; 435/69.4; 435/320.1; 435/325; 530/399; 530/388.24; 536/23.5

(57) ABSTRACT

Novel parathyroid hormone polypeptides and biologically active fragments thereof are disclosed along with nucleic acid molecules encoding same. In particular, parathyroid hormone polypeptides and biologically active fragments (and encoding nucleic acid molecules) derived from fish species (eg Japanese pufferfish (Fugu rubripes)) are disclosed. Such polypeptides and fragments may be used for treatment of diseases associated with abnormal calcium homeostasis (eg osteoporosis, osteopenia, Paget's disease, bone cancer, hyperparathyroidism, hypoparathyroidism, hypercalcemia, psoriasis and other skin-related conditions).

### FIELD OF THE INVENTION

The present invention relates to parathyroid hormone and fragments thereof. More particularly the present invention relates to parathyroid hormone nucleic acid;sequences and polypeptides derived from non-mammalian sources.

### BACKGROUND OF THE INVENTION

Osteoporosis is a leading cause of disability in the elderly, affecting both men and women. Osteopprosis is a progressive disease which results in the reduction of total bone mass and increased fragility. These changes in bone can result in spontaneous fractures of load-bearing bones including hip and vertebrae. These fractures, combined with the increased fragility caused by the disease, can contribute significantly to the physical and mental deterioration of the afflicted individual. Osteoporosis that arises post-menopausally is generally caused by the reduced levels of estrogens characteristic of menopause. Reduced levels of estrogen result in an acceleration of bone turnover with an increased imbalance between resorption of old bone and formation of new bone. This results in thinning, increased porosity, and trabecular depletion of load-bearing bones. Osteoporosis is also associated with other disease states including steroid use, hyperthyroidism, hyperparathyroidism and Cushing's syndrome.

Parathyroio hormone (PTH) is produced by the parathyroid gland and is a major regulator of calcium homeostasis. The principle target cells of PTH occur in bone and kidney. When serum calcium is reduced to below a normal level, the parathyroid gland releases PTH and the calcium level is increased by resorption of bone calcium, increased renal resorption of calcium in the kidney tubules, and via the indirect action of PTH on the intestine to increase absorption of calcium. Although PTH infused continuously at low levels can remove calcium from the bones, the same low doses, can promote bone growth when intermittently injected, therefore suggesting a potential role in the treatment of osteoporosis.

Human PTH (hPTH) is synthesised in parathyroid cells as a 115-amino acid preproparathyroid hormone form, of which 25 amino acids are cleaved off to produce proparathyroid hormone before a further 6 amino acids are cleaved off to result in the 84 amino acid mature form of HPTH. Most of the activity of hPTH resides in the N-terminal 1 to 34 amino acids.

The intracellular pathways involved in mediating the effects of PTH have been elucidated. In renal and osteoblastic cell lines, PTH triggers several parallel intracellular signalling responses, including activation of adenylate cyclase (AC), protein kinase A (PKA), phospholipase C (PLC) and protein kinase C (PKC) and generation of second messengers such as cyclic AMP (cAMP), inositol triphosphate (IP₃), diacylglycerol and increased cytosolic free calcium (Ca²⁺). The activation of PLC results in stimulation of membrane-bound PKC activity. Various groups have considered that the structural determinants for activation of AC/PKA signalling are distinct from those required for activation of PLC or PKC and that these reside, respectively, within the N- and C-terminal domains of PTH(1-34). In particular, the region hPTH(29-32) has been identified specifically as a critical PKC activation domain. It has also been established that the increase in bone growth (ie that effect which is useful in the treatment of osteoporosis), is coupled to the ability of the peptide sequence to increase AC activity. The native hPTH (1-34) sequence has been shown to have all of these activities.

To date, three structurally related but distinct species of PTH receptors have been cloned, PTH-1R and PTH-2R and a third PTH receptor, PTH3R from zebrafish Juppner, et al. Receptors for Parathyroid Hormone and Parathyroid Hormone-related Peptide: Exploration of Their Biological Importance. In Bone, Vol 25 No. 1, July 1999:87-90). The first of these, mammalian PTH-1R, was isolated from both bone and kidney cells and shown to transduce multiple signalling response to PTH(1-34) or parathyroid hormone-related protein (PTHrP) (1-36) when heterologously expressed in cells that lack endogenous PTH-1R. The role of the mammalian PTH-2 R has not been defined. This receptor is activated specifically by PTH and not by PTHrP. Previous efforts to define the contributions of specific regions of the PTH molecule to its binding and signalling properties have been undertaken mainly by use of complex in vivo bioassays, organ cultures, isolated cell membranes or cell lines, generally of rodent origin. Three cDNAs encoding distinct PTH-PTHrP receptors have been identified from zebrafish. Two of these putative receptors appear to be the fish homologs of the PTH1R and PTH2R while the third encodes the novel receptor protein, PTH3R (Rubin, et al, J Biol Chem, 274:28185-28190 (1999), Rubin and Juppner, Isolation and characterization of a novel parathyroid hormone-related peptide (PTHrPrp)-selective receptor and the homolog of the mammalian Parathyroid Hormone (PTH/ PTHrP) Receptor (PTH1R) from Zebrafish. In Danks, J., Dacke, C., Flik, G., and Gay. C, Calcium Metabolism: Comparative Endocrinology. Bristol: BioScientifica. 1999:1-6).

It is known that hPTH and certain analogues of hPTH are stimulators of bone growth that are useful in the treatment of osteoporosis. Although hPTH(1-84) and hPTH(1-34) are promising candidates for treating osteoporosis and other diseases in humans, there is evidence of some associated problems, such as hypercalcemia. Therefore, there is an ongoing need to identify or produce variants of hPTH that provide the biological activity of hPTH but with minimal or reduced clinical side effects.

### SUMMARY OF THE INVENTION

Although parathyroid hormone (PTH) is the main hypercalcemic hormone in mammals, until now it has not been thought to be present in any animal before amphibians in the evolutionary tree as these are the first animals to possess distinct parathyroid glands. The present applicants have isolated a polypeptide from Fugu rubripes with potential to play an important therapeutic role in calcium metabolism and homeostasis in mammals.

Accordingly, in a first aspect the present invention provides a substantially purified polypeptide or biologically active fragment thereof, wherein said polypeptide comprises an amino acid sequence with at least 45% sequence identity with the amino acid sequence set forth in SEQ ID NO: 1.

In a second aspect, the present invention provides an isolated nucleic acid molecule encoding the polypeptide or biologically active fragment of the first aspect.

In a third aspect, the present invention provides a recombinant host, wherein the recombinant host includes a nucleic acid molecule encoding the polypeptide or biologically active fragment of the first aspect.

In a fourth aspect, the present invention provides a pharmaceutical composition comprising the polypeptide or biologically active fragment of the first aspect or a nucleic acid molecule of the second aspect, optionally in combination with a pharmaceutically-acceptable carrier.

In a fifth aspect, the present invention provides a method of treating a disease associated with abnormal calcium homeostasis in a subject, the method comprising administering to the subject a therapeutically effective amount of a polypeptide or biologically active fragment of the first aspect, a nucleic acid molecule of the second aspect, or a pharmaceutical composition of the fourth aspect.

In a sixth aspect, the present invention provides a method of treating a disease that results from altered or excessive action of a PTH receptor, the method comprising administering to a subject a therapeutically effective amount of the polypeptide or biologically active fragment of the first aspect, a nucleic acid molecule of the second aspect, or a pharmaceutical composition of the fourth aspect.

In a seventh aspect, the present invention provides a method for determining rates of bone formation, bone resorption and/or bone remodelling comprising administering to a subject an effective amount of the polypeptide or biologically active fragment of the first aspect labelled with a suitably-detectable label, and determining the uptake of said polypeptide or biologically active fragment into the bone of said subject.

In an eighth aspect, the present invention provides an antibody, wherein the antibody specifically binds to the polypeptide or biologically active fragment of the first aspect.

### BRIEF DESCRIPTION OF THE FIGURES

FIG. 1 shows the nucleotide sequence of a 244bp Fugu PTH DNA clone with the positions of the forward and reverse primers highlighted (in bold and boxed) (SEQ ID NO: 3).

FIG. 2 shows the amino acid sequence of Fugu PTH(1-80) (SEQ ID NO: 1).

FIG. 3 shows the nucleotide sequence of Fugu PTH gene (SEQ ID NO: 2).

FIG. 4 provides a multiple sequence alignment of the deduced amino acid sequence of Fugu PTH(1-80) and human PTH(1-84), chicken PTH(1-88), Fugu PTHrP, sparus PTHrP (AF197094) and human PTHrP (pl22⁷²). Identical residues are shaded in black or grey for PTH and PTHrP, respectively.

FIG. 5(A) shows the adenylate cyclase response of Fugu PTH(1-34), Fugu PTHrP(1-34), human PTH(1-34) and human PTHrP(1-34)on UMR106.01 cells; (B) shows the adenylate cyclase response of Fugu PTH(1-34), Fugu PTHrP(1-34), Fugu PTH(1-26), Fugu PTH(1-29), Fugu PTH(2-34), Fugu PTH(1-32) and Fugu PTH(7-34). The results shown are the mean ±SE of the triplicates. The ID50 of Fugu PTH(1-34) is 15 nM, Fugu PTHrP(1-34) is 1.5 nM and Fugu PTH(1-32) is 9 nM. Both Fugu PTH(1-34) and Fugu PTH(1-32) achieved a higher amplitude of maximum cAMP response than Fugu PTHrP(1-34). Fugu PTH(2-34) was effective to stimulate a cAMP response only when 100 nM or higher was used; (C) shows the adenylate cyclase response when 5 nM and 500 nM of Fugu PTH(1-29) were co-incubated separately in the presence and absence of 5 nM and 100 nM of Fugu PTH(1-34). No antagonist effect was detected. The cAMP response observed has the same amplitude as that of Fugu PTH(1-34) since Fugu PTH(1-29) itself does not appear to be effective to stimulate the adenylate cyclase response; (D) shows the adenylate cyclase response when 5 nM and 500 nM of Fugu PTH(2-34) were co-incubated separately in the presence and absence of 5 nM and 100 nM of Fugu PTH(1-34). No antagonist effect was detected and, when 100 nM Fugu PTH(1-34) was co-incubated with 500 nM Fugu PTH(2-34), a cAMP response was observed which was greater than the sum of the effect of the two peptides when tested separately; (E) shows the adenylate cyclase response when 5 nM and 500 nM of Fugu PTH(7-34) were co-incubated separately in the presence and absence of 5 nM and 100 nM of Fugu PTH(1-34). No antagonist effect was detected. The cAMP response observed when 100 nM Fugu PTH(1-34) was co-incubated with 500 nM Fugu PTH(7-34) was greater than the sum of the effect of the two peptides when tested separately.

FIG. 6 shows stained sections of proximal tibiae from (from left to right) untreated rats and rats treated with Fugu PTH(1-34) showing that Fugu PTH(1-34) increases bone mass in young rats. In particular, the figure shows toluidine blue stained sections of proximal tibiae from untreated control rats and rats treated with 3 microgram/100 gram body weight/day Fugu PTH(1-34) (“low dose fPTH”), 10 microgram/100 gram body weight/day Fugu PTH(1-34) (“high dose fPTH”), 3 microgram/100 gram body weight/day human PTH (“low dose hPTH”) and 10 microgram/100 gram body weight/day human PTH (“high dose hPTH”), and an apparent elevation of trabecular density in rats treated with Fugu PTH(1-34).

FIG. 7 shows histomorphometric analysis of the proximal secondary spongiosa revealing a significant increase in trabecular bone volume (% of total volume), trabecular thickness (μm), and trabecular number (per mm) without a reduction in trabecular separation (μm) in rats treated with high dose Fugu PTH(1-34) (10 microgram/100 gram body weight/day). Values are mean+SEM for each group, n=5-8 rats per group. *, p<0.05; **, p<0.01; ***, p<0.001 vs untreated control by one-way ANOVA followed by Tukey's post hoc test.

FIG. 8 shows sections of the proximal tibiae, selected from different treatment groups to represent the mean trabecular bone volume determined by histomorphometry. The sections were stained with a modified von Kossa stain which stains calcified matrix black, and with a Ponceau/Orange G counterstain of the marrow. The box in the section shown on the left of the figure represents the region chosen for histomorphometric analysis. The treatment groups represented are (from left to right) untreated control rats and rats treated with 3 microgram/100 gram body weight/day Fugu PTH(1-34), 10 microgram/100 gram body weight/day Fugu PTH(I-34), 3 microgram/100 gram body weight/day human PTH and 10 microgram/100 gram body weight/day human PTH.

FIG. 9 shows, graphically, that histomorphometric markers of bone formation were significantly elevated by human PTH (hPTH) and, to a lesser extent by high dose Fugu PTH(1-34) (10 microgram/100 gram body weight/day) treatment. The increases in both osteoblast number and surface suggest an increase in osteoblast proliferation in response to Fugu PTH(1-34), as observed with human PTH (hPTH); low dose Fugu PTH(1-34) (3 microgram/100 gram body weight/day) resulted in a trend towards increased osteoblast proliferation. Values are mean+SEM for each group, n=5-8 rats per group. *, p<0.05; **, p<0.01; ***, p<0.001 vs untreated control by one-way ANOVA followed by Tukey's post hoc test.

FIG. 10 shows, graphically, that the osteoblast number per unit osteoid surface was significantly increased by both low (3 microgram/100 gram body weight/day) and high (10 microgram/100 gram body weight/day) doses of Fugu PTH(1-34), and by the highest (10 microgram/100 body weight/day) dose of human PTH (hPTH). Values are mean+SEM for each group, n=5-8 rats per group. *, p<0.05; * p<0.01; ***, p<0.001 vs untreated control by one-way ANOVA followed by Tukey's post hoc test.

FIG. 11 shows, graphically, that histomorphometric markers of bone resorption were significantly reduced by high dose human PTH (hVFH) (10 microgram/100 gram body weight/day), but not significantly altered by Fugu PTH, although there is a trend towards reduced osteoclast number in animals given high dose (10 microgram/100 gram body weight/day) Fugu PTH(1-34). Values are mean+SEM for each group, n=5-8 rats per group. *, p<0.05; **, p<0.01; ***, p<0.001 vs untreated control by one-way ANOVA followed by Tukey's post hoc test.

FIG. 12 shows the nucleotide sequence of gummy shark PTH gene (SEQ ID NO: 4).

FIG. 13 shows an alignment of three possible translations of the gummy shark PTH gene against the amino acid sequences of Fugu PTH and human PTH.

### DETAILED DESCRIPTION OF THE INVENTION

The applicants have isolated and sequenced a PTH-like gene from Fugu rubipes.

Accordingly, in a first aspect the present invention provides a substantially purified polypeptide or biologically active fragment thereof, wherein said polypeptide comprises an amino acid sequence with at least 45% sequence identity with the amino acid sequence set forth in SEQ ID NO: 1.

The term “polypeptide” as used herein refers to any peptide, polypeptide or protein comprising two or more amino acids joined to each other by peptide bonds or modified peptide bonds, ie peptide isosteres. Such “polypeptides” may contain amino acids other than the 20 gene-encoded amino acids and comprise amino acid sequences modified either by natural processes, such as post-translational processing, or by chemical modification techniques which are well known to persons skilled in the art. Modifications can occur anywhere in such polypeptides, including the peptide backbone, the amino acid side-chains and/or the amino or carboxyl termini. It will also be appreciated that the same types of modifications may be present in the same or at varying degrees at several sites in such polypeptides. Further, such polypeptides may contain many types of modifications (see, for instance, Proteins-Structure and Molecular Properties, 2nd Ed., T E Creighton, W H Freeman and Company, New York, 1993; and Wold, F, Posttranslational Protein Modifications: Perspectives and Prospects, pgs 1-12 in Posttranslational Covalent Modification of Proteins, B C Johnson, Ed, Academic Press, New York, 1983; Seifter et al,

“Analysis for protein modifications and nonprotein cofactors”, Methods in Enzymology 182:626-646 (1990); and Rattan et al, “Protein Synthesis: Posttranslational Modifications and Aging”, Ann N Y Acad Sci, 663:48-62 (1992). Peptides, polypeptides and proteins included within the term “polypeptide” as used herein, may be isolated from suitable sources, synthesised using techniques well known to persons skilled in the art, produced by recombinant techniques well known to persons skilled in the art, or otherwise obtained from suitable commercial sources.

Biologically active fragments encompassed by the present invention include fragments that lack at least one amino acid of the amino acid sequence set forth in SEQ ID NO: 1 yet retain at least one of the biological activities characteristic of a polypeptide comprising the amino acid sequence set forth in SEQ ID NO: 1. For example, the fragment can activate adenylate cyclase and result in cAMP accumulation when used in an assay for adenylate cyclase activity (eg the assay described in Example 1 herein).

In a preferred embodiment, the present invention provides a substantially purified polypeptide, or a biologically active fragment thereof, wherein the polypeptide comprises an amino acid sequence with at least 55% sequence identity with the amino acid sequence set forth in SEQ ID NO: 1. More preferably, the polypeptide comprises an amino acid sequence with at least 65% sequence identity with the amino acid sequence set forth in SEQ ID NO: 1. Still more preferably, the polypeptide comprises an amino acid sequence with at least 75% sequence identity with the amino acid sequence set forth in SEQ ID NO: 1. Even more preferably, the polypeptide comprises an amino acid sequence with at least 90% sequence identity with the amino acid sequence set forth in SEQ ID NO: 1. Most preferably, the polypeptide comprises an amino acid sequence with at least 95% sequence identity with the amino acid sequence set forth in SEQ ID NO: 1.

In a further preferred embodiment, the biologically active fragment of the present invention comprises an amino acid sequence with at least 65% sequence identity with the sequence at amino acids 1-34 of SEQ ID NO: 1. More preferably, the biologically active fragment comprises an amino acid sequence with at least 75%, more preferably at least 90%, and most preferably 95%, sequence identity with the sequence at amino acids 1-34 of SEQ ID NO: 1.

In a still further preferred embodiment, the biologically active fragment of the present invention comprises an amino acid sequence with at least 65% sequence identity with the sequence at amino acids 7-34 of SEQ ID NO: 1. More preferably, the biologically active fragment comprises an armino acid sequence with at least 75%, more preferably at least 90%, and most preferably 95%, sequence identity with the sequence at amino acids 7-34 of SEQ ID NO: 1.

The term “sequence identity” as used herein refers to a measure of the identity of amino acid sequences wherein the sequences are aligned so that the highest order match is obtained, and which can be calculated using published techniques or methods codified in computer programs such as, for example, BLASTP, BLASTN, FASTA (Atschul et al, J Molec Biol, 215:403 (1990)).

In a most preferred embodiment of the biologically active fragment of the present invention, the biologically active fragment consists of an amino acid sequence which substantially corresponds to the sequence of amino acids 1-34 or 7-34 of SEQ ID NO: 1.

Further, the polypeptide or biologically active fragment of the present invention comprises an amino acid sequence with threonine at position 1 (ie Thr 1). Preferably, the polypeptide or biologically active fragment comprising threonine at the N-terminus is derived from a bony or cartilaginous fish species.

Also contemplated by the present invention are polypeptides or biologically active fragments thereof comprising a hybrid amino acid sequence derived from parathyroid hormone or parathyroid-like hormone polypeptides from different species. For example, polypeptides or biologically active fragments thereof comprising a hybrid amino acid sequence derived from a parathyroid-like hormone polypeptide from a bony or cartilaginous fish (eg Fugu PTH) and human and/or other mammalian and/or avian parathyroid hormone polypeptide. Such hybrid polypeptides or biologically active fragments thereof preferably comprise threonine at the N-terminus.

The term “substantially corresponding” as used herein in relation to the amino acid sequence of the polypeptide or biologically active fragment of the present invention, is intended to encompass the exact amino acid sequence as well as minor variations which do not result in a substantial decrease in the biological activity of the amino acid sequence (eg variations which do not diminish the ability of the polypeptide or biologically active fragment to activate adenylate cyclase and result in cAMP accumulation when used in an assay for adenylate cyclase activity). These variations may include one or more conservative amino acid substitutions. The conservative amino acid substitutions envisaged are: G, A, V, I, L, M; D, E, N, Q; S, C, T; K, R, H; and P, Nα-alkylamino adds.

In a second aspect, the present invention provides an isolated nucleic acid molecule encoding the polypeptide or biologically active fragment of the first aspect of the invention.

In a preferred embodiment, the nucleic acid molecule comprises a nucleotide sequence substantially corresponding to the nucleotide sequences set forth in SEQ ID NO: 2 or a fragment thereof.

In a preferred embodiment, the nucleic acid molecule of the second aspect is inserted into a cloning or expression vector.

Cloning vectors for use with the present invention include plasmid or phage DNA or other DNA vectors which are able to replicate autonomously in a host cell. The cloning vector may further comprise a selectable marker suitable for use in the identification (ie selection) of cells transformed with the cloning vector. Suitable markers include those which, for example, provide tetracycline resistance or ampicillin resistance.

Expression vectors for use with the present invention include vectors similar to cloning vectors but which are capable of enhancing the expression of a gene which has been cloned into it, after transformation into a host. Cloned genes will usually be placed under the control of (ie operably linked to) certain control sequences such as promoter sequences. Suitable promoter sequences include both constitutive and inducible promoter sequences.

The term “nucleic acid molecule” as used herein includes any polyribonucleotide or polydeoxribonucleotide, and which may be single- or double-stranded and therefore includes single- and double-stranded DNA, DNA that is a mixture of single- and double-stranded regions, single- and double-stranded RNA, and RNA that is mixture of single- and double-stranded regions, and hybrid molecules comprising DNA and RNA that may be single-stranded or, more typically, double-stranded or a mixture of single- and double-stranded regions. In addition, the term “nucleic acid molecule” also includes DNA and RNA containing one or more modified bases and DNA and RNA with backbones modified for stability or for other reasons. “Modified” bases include, for example, tritylated bases and unusual bases such as inosine. The term “nucleic acid molecule” also embraces relatively short polynucleotides, often referred to as oligonucleotides.

In a third aspect, the present invention provides a recombinant host, wherein the recombinant host includes a nucleic acid molecule encoding the polypeptide or biologically active fragment of the first aspect.

Suitable recombinant hosts include any prokaryotic or eukaryotic host cells which contain include the nucleic acid molecule within, for example, a cloning vector or expression vector, as well as any prokaryotic or eukaryotic host cells which have been genetically engineered to include the desired nucleic acid molecule in the host chromosome or genome. Representative examples of appropriate host cells include bacterial cells, such as streptococci, staphylococci, E coli, Streptomyces and B. sublilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells (see Sambrook et al., Molecular Cloning: A Laboratory Manual, Second Edition, Cold Spring Harbor Laboratory, New York (1989)).

Preferred recombinant hosts are eukaryotic cells transformed with a cloning vector or expression vector including the nucleic acid molecule of the present invention. More specifically, recombinant mammalian cells transformed with a cloning vector or expression vector including the nucleic acid molecule of the present invention are preferred.

Suitable recombinant hosts also include transgenic animals, all of whose germ and somatic cells include the nucleic acid molecule of tne present invention. Such transgenic animals will typically be vertebrates, particularly mammals such as non-human primates, mice, sheep, pigs, cattle, goats, guinea pigs, rodents (eg mice and rats), and the like.

Introduction of the nucleic acid molecule of the present invention into host cells can be effected by techniques well known to persons skilled in the art (eg those described in many standard laboratory manuals, such as Davis et al, Basic Methods in Molecular Biology (1986) and Sambrook et al, 1989 supra including calcium phosphate transfection, DEAE-dextran mediated transfection, transvection, microinjection, cationic lipid-mediated transfection, electroporation, transduction, scrape loading, ballistic introduction and infection).

Recombinant hosts of the present invention may be used for the recombinant production of the polypeptide or biologically active fragment of the first aspect.

In a fourth aspect, the present invention provides a pharmaceutical composition comprising a polypeptide or biologically active fragment of the first aspect or a nucleic acid molecule of the second aspect, optionally in combination with a pharmaceutically-acceptable carrier.

In a fifth aspect, the present invention provides a method of treating a disease associated with abnormal calcium homeostasis in a subject, the method comprising administering to the subject a therapeutically effective amount of the polypeptide or biologically active fragment of the first aspect, a nucleic acid molecule of the second aspect, or a pharmaceutical composition of the fourth aspect.

Preferably, the disease is selected from the group including bone fractures, osteoporosis, Paget's disease, bone cancer (including secondary cancer in bone resulting from a primary tumor elsewhere), hyperparathyroidism, hypoparathyroidism, psoriasis and other skin-related conditions.

A pharmaceutical composition comprising the polypeptide or biologically active fragment of the present invention are useful for the prevention and treatment of a variety of mammalian conditions resulting from alterations in calcium homeostasis and include those manifested by loss of bone mass. Thus, the pharmaceutical composition of the present invention, in particular, are indicated for the prophylaxis and therapeutic treatment of osteoporosis and osteopenia in humans.

Further, the pharmaceutical composition of the present invention are indicated for the prophylaxis and therapeutic treatment of other bone diseases, including the prophylaxis and therapeutic treatment of hypoparathyroidism.

Still further, the pharmaceutical composition of the present invention are indicated for use as agonists for fracture repair and as antagonists for hypercalcemia.

Some forms of hypercalcemia and hypocalcemia are related to the interaction between PTH and PTHrP and the PTH-1R, PTH-2R and/or PTH3R receptors. Hypercalcemia is a condition in which there is an abnormal elevation in serum calcium level and is often associated with other diseases, including hyperparathyroidism, osteoporosis, carcinomas of the breast, lung and prostate, epidermoid cancers of the head and neck and of the esophagus, multiple myeloma, and hypernephroma. On the other hand, hypocalcemia is a condition in which the serum calcium level is abnormally low, and may result from a deficiency of effective PmH (eg following thyroid surgery).

Typically, the pharmaceutical composition of the present invention will be administered to a subject in an amount providing between about 0.01 and about 100 microgram/kilogram body weight per day of the active (ie the polypeptide or biologically active fragment of the present invention), more preferably providing from 0.05 and 25 microgram/kilogram body weight per day of the active, and most preferably providing from about 0.07 to about 1.0 microgram/kilogram body weight per day of the active. For a 50 kilogram human female subject, the daily dose of the active will therefore be from about 0.5 to about 500 micrograms, more preferably from about 2.5 to about 125 micrograms, most preferably from about 3.5 to about 50 micrograms. In other mammals, such as horses, dogs, and cattle, higher doses may be required. This dosage may be delivered in a pharmaceutical composition intended for a single daily administration, multiple daily administration, or controlled or sustained release, as needed to achieve the most effective results, or more preferably, by injection (by any of the subcutaneous, transcutaneous, intramuscular and intravenous routes) one or more times daily. Most preferably, this dosage may be delivered in a pharmaceutical composition intended for nasal insufflation.

The selection of the exact dosage and the most appropriate delivery regimen will be influenced by, inter alia, the pharmacological properties of the selected active (ie the polypeptide or biologically active fragment of the present invention), the nature and severity of the disease or condition being treated, and the physical condition and mental acuity of the subject.

The active (ie the polypeptide or biologically active fragment of the present invention) may be present in the pharmaceutical composition of the present invention in the form of a pharmaceutically acceptable salt which retains the desired biological activity without toxic side effects. Examples of such salts are: (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, naphthalenesulfonic acids, naphthalene disulfonic acids, polygalacturonic acid and the like; (b) base addition salts formed with polyvalent metal cations such as zinc, calcium, bismuth, barium, magnesium, aluminium, copper, cobalt, nickel, cadmium, and the like; or with an organic cation formed from N,N′-dibenzylethylenediamine or ethylenediamine; and (c) combinations of (a) and (b), for example, a zinc tannate salt and the like.

As mentioned above, one preferred route of administration for the pharmaceutical composition is by nasal insufflation. A pharmaceutical composition for such administration may comprise a surfactant acid to enhance the absorption of the active across the nasal mucous membrane. Suitable surfactant acids include, for example, glycocholic acid, cholic acid, taurocholic acid, ethocholic acid, deoxycholic acid, chenodeoxycholic acid, dehydrocholic acid, glycodeoxycholic acid, cyclodextrins. Such surfactant acids may be included in the pharmaceutical composition in an amount in the range between about 0.2 and about 15 weight percent, preferably between about 0.5 and about 4 weight percent, most preferably about 2 weight percent.

Like PTH, the polypeptide or biologically active fragment of the present invention may be administered in combination with-other agents useful in treating a given clinical condition. For example, for the treatment of osteoporosis and other bone-related disorder, the polypeptide or biologically active fragment may be administered in conjunction with a dietary calcium supplement or with a vitamin D analogue (see U.S. Pat. No 4,698,328). Alternatively, the polypeptide or biologically active fragment may be administered, preferably using a cyclic therapeutic regimen, in combination with bisphosphonates as described in U.S. Pat. No 4,761,406, or in combination with one or more bone therapeutic agents such as calcitonin and estrogen, or in combination with Raloxifene and other related selective estrogen receptor modulator (SERM) drugs.

In a sixth aspect, the present invention provides a method of treating a disease that results from altered or excessive action of a PTH receptor, the method comprising administering to a subject a therapeutically effective amount of the polypeptide or biologically active fragment of the first aspect, a nucleic acid molecule of the second aspect, or a pharmaceutical composition of the fourth aspect.

In a seventh aspect, the present invention provides a method for determining rates of bone formation, bone resorption and/or bone remodelling comprising administering to a subject an effective amount of the polypeptide or biologically active fragment of the first aspect labelled with a suitably-detectable label, and determining the uptake of said polypeptide or biologically active fragment into the bone of said subject.

In a preferred embodiment, the polypeptide or biologically active fragment is labelled with a label selected from the group consisting of: radiolabel, fluorescent label, bioluminescent label. More preferably, the polypeptide or biologically active fragment is labelled with 99Technicium.

In an eighth aspect, the present invention provides an antibody, wherein the antibody specifically binds to the polypeptide or biologically active fragment of the first aspect.

The polypeptide or biologically active fragment of the present invention may be used to produce both monoclonal and polyclonal antibody reagents, by any of the techniques well known to persons skilled in the art. For example, antibody reagents may be prepared by immunising appropriate host animals with the polypeptide or biologically active fragment of the present invention either alone or in the presence of adjuvants and/or carrier proteins. Examples of appropriate hosts include mice, rats, rabbits, sheep, horses, goats and cows. For the production of monoclonal antibody reagents, the techniques that can be employed include that set out by Kohler et al, Eur J Immunol, 6:11-19 (1976).

In order that the nature of the present invention may be more clearly understood, preferred forms thereof will now be described with reference to the following non-limiting examples.

### EXAMPLE 1

Identification of a Parathyroid Hormone in the Fish, Fugu Rubipes.

Materials and Methods

Polymerase Chain Reaction and Automated Sequencing of a DNA Clone Encoding Fugu PTH(1-80)

Primers for polymerase chain reaction (PCR) were designed from some preliminary data obtained from Joint Genome Institute (http://www.jgi.doe.gov/programs/fugu.htm) using known PTH amino acid sequences. The preliminary nucleic acid sequence obtained from the database was checked by PCR and a number of mistakes determined. New PCR primers were designed to the revised nucleic acid sequence; forward primer-[5′-CAGTGAGTGAAGTCCAGCTCA-3′] (SEQ ID NO: 5) and reverse primer-[5′-CTTCACTCCTGTGATTTGAGCA-3′] (SEQ ID NO: 6). PCR amplification was performed on approximately lOng genomic DNA isolated from Fugu rubripes. PCR products were purified using a commercially available kit (UltraClean PCR Clean-Up DNA Purification Kit, Geneworks, Adelaide, Australia) and DNA was sequenced using the ABI Prism BigDye Terminator Cycle Sequencing Ready Reaction Kit (Perkin Elmer, Boston, USA).

Multiple sequence alignments were carried out using ClustralW (Thompson et al., . Nucleic Acids Res, 22:4673-4680 (1994)) and displayed in PrettyBox (Rick Westerman, Purdue University). Percentage identities and similarities were calculated using Gap (Henikoff and Henikoff, Proc Natl Acad Sci USA, 98:10915-10919 (1992)).

Synthetic Peptides

The N-terminus region of the protein Fugu PTH(1-34) and various fragments (ie Fugu PTH(1-26), Fugu PTH(1-29), Fugu PTH(1-34), Fugu PTH(2-34), Fugu PTH(1-32) and Fugu PTH(7-34)) as well as the N-terminus of Fugu PTHrP(1-34) were synthesised using an Applied Biosystems 433A peptide synthesiser (Foster City, USA) using Rink resin and Fmoc chemistry with Fastmoc 0.1 Dry Conditions monitor. The completed peptides were simultaneously deprotected and cleaved from the resin (cleavage was carried out in 82.5% trifluroacetic acid with Reagent K (Auspep, Parkville, Australia) consisting of 5% ophenol, 5% water, 5% thioanisole and 2.5% ethandithiol). The peptides were extracted from the resin in 20% (v/v) acetonitrile and 0.1% (v/v) trifluroacetic acid, dried down. The peptides were purified by sequential ion-exchange chromatography (MacS) with 20% (v/v) acetonitrile (Mallinkrodt HPLC grade, St Louis, USA) and 0.1% (v/v) trifluroacetic acid using a gradient of 0-1M guanidine hydrochloride. The fractions were then checked by mass spectrometry and pooled. The pooled samples were purified by preparative low pressure reversed phase chromatography (25×400 column, C18, 250 Ångstrom, 35 to 70 micrometre Amicon resin) with an acetonitrile gradient in the presence of 0.1% (v/v) trifluroacetic acid. Mass spectrometry verified the purity of the synthetic peptides (PerSephive Biosystems Voyager DE,(Foster City, USA) with Data Explorer Software Version 4.0). The synthetic Fugu PTH(1-34) and Fugu PTHrP(1-34) were analysed by nanospray mass spectrometry (Applied Systems QSTAR pulsar, Foster City, USA).

Biological Activity

The PTH-like biological activity of the peptides was assayed by measuring cyclic adenosine 3′,5′-monophosphate (cAMP) production in UMR106.01 cells (Forrest et al, Calcif Tissue Int, 37:52-56 (1985)), grown to 90% confluence. Prior to assaying, the cells were washed once with phosphate buffered saline (PBS) and equilibrated for 20 mins in medium containing 0.1% BSA and lmM-isobutylmethylxanthine (Sigma, St Louis, USA). Cells were subsequently stimulated at 37° C for 10 mins in the absence and presence of increasing hormone concentrations. The cells were then washed once with PBS and cAMP was extracted with 1.5 ml acidified ethanol. Samples were evaporated to dryness, reconstituted in assay buffer and assayed by a specific cAMP radioimmunoassay (Houssami et al, Endocrine J. 2:127-134 (1994)).

Immunoblots

Seven antisera (R88, R1904, R1942, R87, R1348, R196, R212) were raised against human PTHrP(1-14) and one antiserum (R190) was raised against human PTHrP(1-141). The anti-PTH polyclonal antibody was raised against hPTH(1-34)(BioGenex, San Ramon, USA). The anti-human PTHrP antisera have been used successfully in immunohistochemistry and Western blotting with tissues taken from bony and cartilaginous fishes (Danks et al, Gen Comp Endocrinol, 92:201-212 (1993); Ingleton et al, Gen Comp Endocrinol 98:211-218 (1995)). The anti-PTH antiserum has been used in immunohistochemistry of human parathyroid material and Western blotting (Danks et al, J Pathol, 161:27-33 (1993)). 10, 25, 50 μg of Fugu PTH were spotted along side a positive control, (ie human PTHrP(I-34)), on nitrocellulose membrane (Schleicher & Schuell, Dassel, Germany). After an initial incubation period with Tris buffered saline, 0.2% Tween, 5% skim milk powder to block non specific binding sites, the nitrocellulose membrane was incubated with primary polyclonal antibody, followed by secondary antibody (anti-rabbit serum conjugated to horseradish peroxidase (Dako, Carpenteria, USA)) incubation. Three washes on a shaker table were done in between incubations. A BM chemiluminescence blotting system (Roche Applied Sciences, Mannheim, Germany) was used to detect specific dot blots.

Results

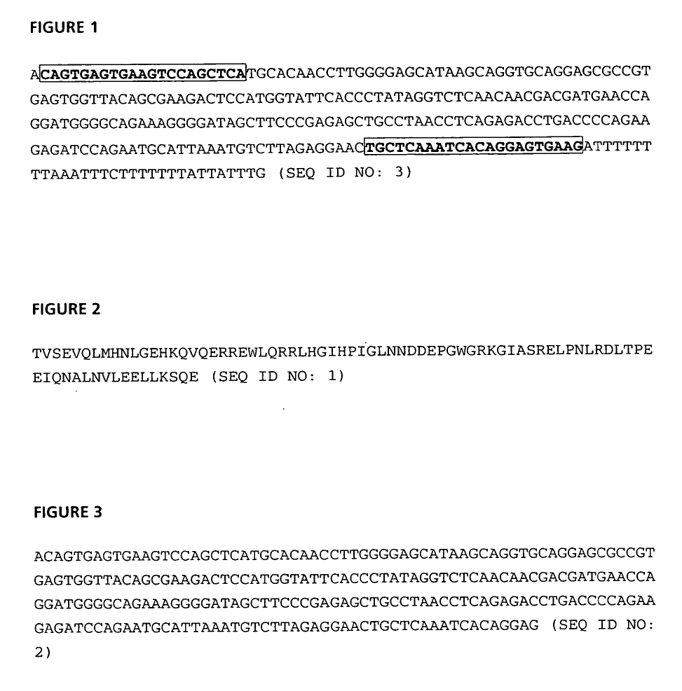

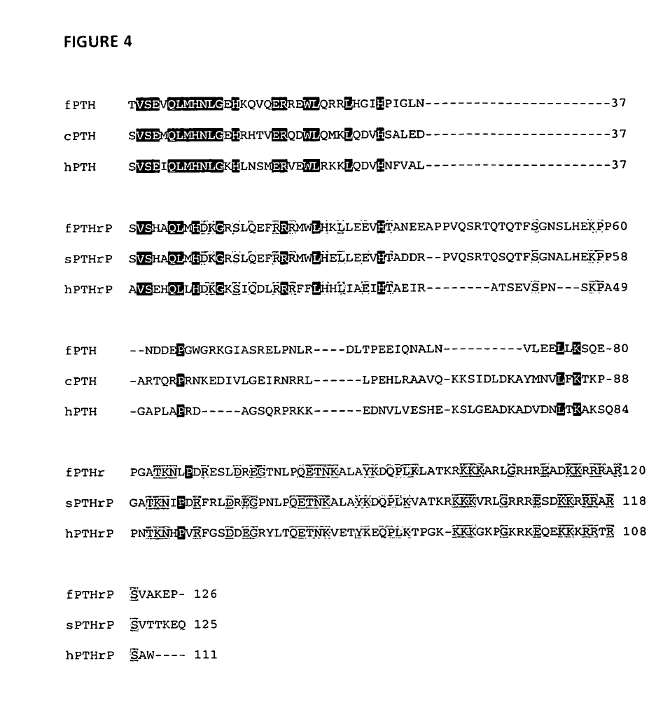

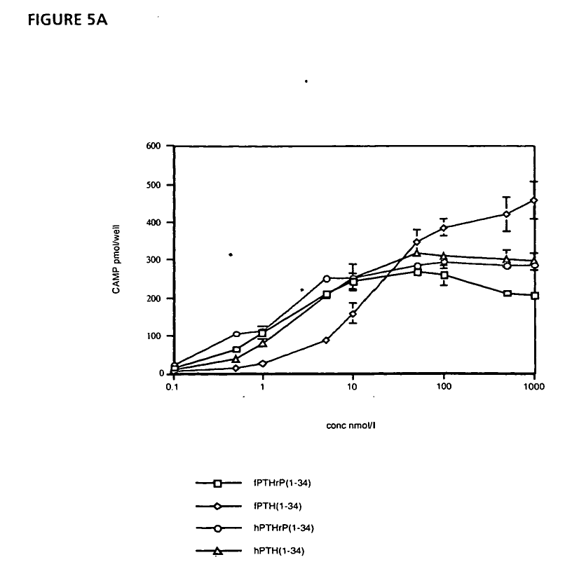

Amplification by PCR yielded a 244 bp product with the nucleic acid sequence shown with the forward and reverse primers in FIG. 1 (see also FIG. 3). Translation of this sequence produces an 80 amino acid sequence (FIG. 2) and shows that the coding region of the Fugu PTH gene has very low overall sequence homology to either chicken PTH or human PTH. The sequence identity between Fugu PTH and chicken PTH is 36% and similarity is 49% and the sequence identity between Fugu PTH and human PTH is 32% while the similarity is 44% (FIG. 4).

The amino acid sequence identity between Fugu PTHrP and human PTHrP is 53% and the similarity is 64%. If only the N-terminal 34 amino acid region is considered, Fugu PTH(1-34) has 56% identity to chicken PTH and 53% to human PTH but the similarity is 68% to chicken PTH and 65% to human PTH. Sequence identity in the N-terminal regions of Fugu PTHrP and human PTHrP is 59%o and the similarity is 77%. These results are summarized in Table 1 (see also FIG. 4).

TABLE 1
Amino acid sequence comparisons of the N-terminal
34 amino acids or the whole protein coding sequence
of Fugu PTH and Fugu PTHrP.

| | cPTH | hPTH | fPTHrP | SPTHrP | hPTHrP |
|---|---|---|---|---|---|
| fPTH(1-34) | 68/56 | 65/53 | 47/41 | 44/41 | 42/38 |
| fPTH(1-80) | 48/37 | 44/33 | 36/29 | 35/30 | 33/27 |
| fPTHrP(1-34) | 50/44 | 55/47 | 100/00 | 97/97 | 76/58 |
| fPTHrP(1-126) | 38/30 | 40/33 | 100/100 | 89/87 | 64/53 |

Percent Similarity/Percent Identity

The purified Fugu PTH(1-34) had a mass of 4,154.75 Da and the purified Fugu PTHrP had a mass 4,126.4529 Da. The fully automated synthesis of Fugu PTHrP(1-34) resulted in the quantative transamidation of Asp1O by piperadine, adding 67 Da (http://www.abrf.org/index.cfm/dm.details?DMID=67&AvgMass=67&Margin=0) but this was overcome by the manual addition of His 9.

The synthetic peptides had their purity checked with mass spectrometry and the resultant traces showed a single dominant peak, indicating that the peptides were of the required length and between 90-95% pure.

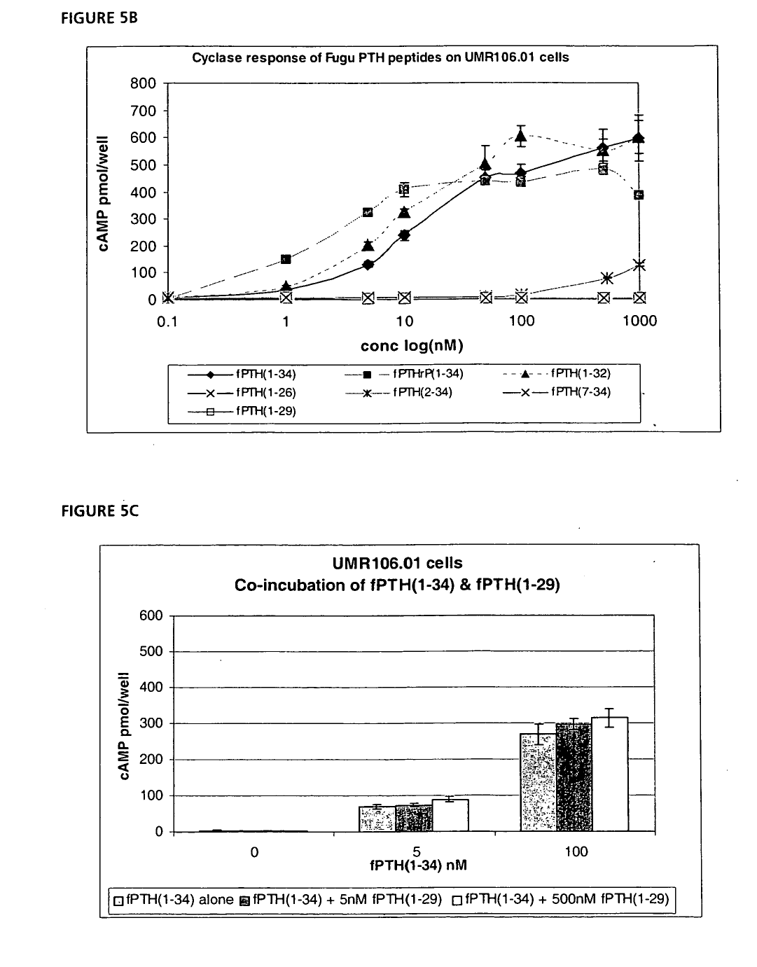

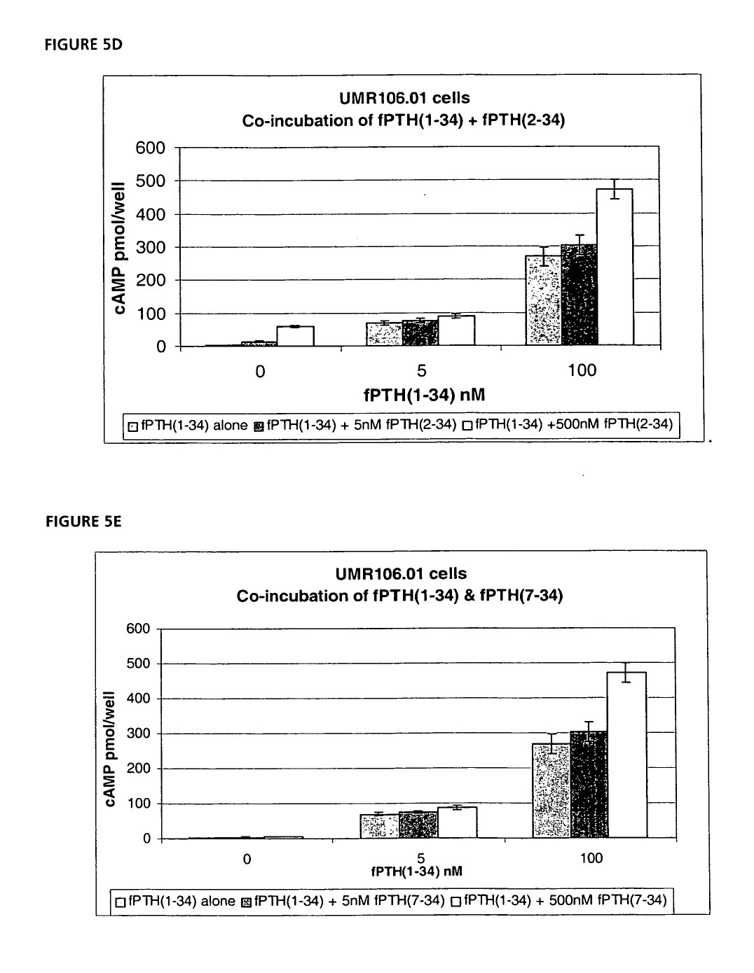

Fugu PTH(1-34) stimulated cAMP formation (ID50=17±3.6 nM, n=4) in a dose-dependent manner with a potency that was consistently less than that of Fugu PTHrP(1-34) (ID50=1.4±0.48 nM, n=4) (FIG. 5A). Fugu PTH(1-34) is less potent than human PTH(1-34), human PTHrP(1-34) and Fugu PTHrP(1-34), Fugu PTH(I-34) (FIG. 5A), but the maximum amplitude of response to Fugu PTH(1-34) was significantly greater than that achieved with the highest concentrations of human PTH, human PTHrP or Fugu PTHrP. Fugu PTH(1-32) also stimulated cAMP formation, while little or no cAMP formation was observed with Fugu PTH(1-26), Fugu PTH(1-29), Fugu PTH(2-34) and Fugu PTH(7-34) alone (FIG. 5B).

When UMR 106.01 cells were co-incubated with 100 nM Fugu PTH(1-34) and a maximal dose of 10 nM of Fugu PTHrP (1-34), no further increase in adenylate cyclase activity was observed consistent with the premise that the two peptides act through the same receptor (data not shown).

When increasing concentrations of human PTHrP(7-34) were co-incubated with 10 nM Fugu PTH(1-34), 1 nM human PTH(1-34), 0.5 nM Fugu PTHrP(1-34) or 0.5 nM human PTHrP(1-34), partial inhibition of the cAMP response was evident with all of the peptides tested (data not shown) (McKee et al, Endocrinol, 122:3008-3010 (1988)), providing further evidence that Fugu PTH(1-34) acts through the PTH-1R.

In further co-incubation assays, the results of which are shown in FIGS. 5C-E, no change in adenylate cyclase activity was observed with Fugu PTH(1-34) (5 nM and 100 nM amounts) and Fugu PTH(1-29) (5 nM and 500 nM amounts), whereas increases (greater than sum) were observed with Fugu PTH(1-34) (100 nM) and Fugu PTH(2-34) (500nM), as wel as with Fugu PTH(1-34) (100 nM) and Fugu PTH(7-34) (500 nM).

The immunoblots showed that Fugu PTH(1-34) does not cross-react with any of the rabbit polyclonal antisera raised against human PTHrP(I-14) or the rabbit polyclonal antiserum that is raised against human PTH(1-34) (data not shown).

Discussion

The N-terminal region of the Fugu PTH polypeptide identified in this example, is homologous with the N-terminus of tetrapod PTH and with PTHrP from both mammals and fish. Eighteen of the first 34 amino acids of Fugu PTH are identical to those in human PTH while 14 of the first 34 amino acids of Fugu PTH are identical to those in Fugu PTHrP. Whereas 20 of the first 34 amino acids of Fugu PTHrP and human PTHrP are identical, only 13 in this region are identical between Fugu PTH and Fugu PTHrP. This suggests that the fish sequence that has been isolated is more like PTH than PTHrP. In the amino acid sequence of Fugu PTH, after the first 34 amino acids, there is no significant homology to either human PTH or chicken PTH.

The biological assay data is consistent with an action of Fugu PTH through the PTH1R, as is the case with human PTH and human PTHrP. Structural analyses using nuclear magnetic reasonance and X-ray crystallography, together with extensive studies of cross-linking of PTH and PTHrP analogs to the PTH1R, are all in accord with a model of PTH and PTHrP binding through participation of residues within the sequence between residue 15 and 31. There are a number of structural aspects of the N-terminal portion of the Fugu PTH molecule, which fit comfortably with what is known of PTH1R interactions. Photoaffinity cross-linking studies have identified certain residues that are crucial for binding of PTH and PTHrP to PTH1R. Residues Phe 23, Leu 24 and Ile 28 are close to the receptor and photolabeling of Leu 24 causes a 10-fold reduction in binding (Censure et al, J Biol Chem, 276:28650-28658 (2001)). When this is considered together with the fact that residues Phe 23, Leu 24 and Ile 28 are intolerant to substitution by polar residues (Gardella et al, Endocrinol, 132:2024-2030 (1993); Gardella et al, J Biol Chem, 271:19888-19893 (1996)), the Fugu PTH is in keeping with that of other PTH and PTHrP homologs. Further, the Arg 20 and Leu 24 of Fugu PTH are consistent with the strict conservation of these residues throughout all known PTH and PTHrP sequences. On the other hand, residues Lys 26, Gin 29 and Asp 30 can be mutated without effect on receptor binding (Gardella et al, 1993 supra).

The potency of Fugu PTH(1-34) shown in this example was consistently about one-fifth to one-tenth that of human PTH or PTHrP. This might be due to subtle conformational changes resulting from the different sequence in the C-terminal portion of Fugu PTH(1-34), for example the fact that all of residues 26, 27, 29 and 30 are variations from those positions in the other PTH/PTHrP homologs. While the reduced potency of Fugu PTH on adenylate cyclase activation on a mammalian target cell is interesting, it remains to be discovered what is the true target in the fish, and the peptide's potency. It may however, be relevant to note that in the PTH-3R discovered in zebrafish (Rubin et al, 1993 supra), activation by human PTH was consistently 20-fold less potent than that either by human PTHrP or Fugu PTHrP.

The potency of Fugu PTH(2-34) was considerably less than Fugu PTH(1-34) indicating that the threonine at the N-terminus (ie position 1) of the Fugu PTH(1-34) peptide is an important residue in conferring biological activity. The cAMP response observed when 100 nM Fugu PTH(1-34) was co-incubated with 500 nM Fugu PTH(2-34) is greater than the sum of the effect of the two peptides when tested separately, indicating that there may be a synergistic effect in having a threonine in position 1 of Fugu PTH (1-34). In contrast, deletion of the two C-terminal amino acids from Fugu PTH(1-34) had a minimal effect on the stimulation of cAMP activity.

Immunologically, Fugu PTH(1-34) is not recognised by any of the human PTH or human PTHrP antisera even at high concentrations of peptide and antisera. It is very unlikely that any antisera raised to human PTHrP, human PTH and bovine PTH could localise the fish PTH homolog in fish tissues since the N-terminus of Fugu PTH is the portion of the polypeptide which is the most highly conserved. The finding that the N-terminus of the Fugu PTH has only 18 of 34 amino acids identical to human PTH with a threonine at position 1 instead of a serine, may determine the lack of cross-reactivity with the polyclonal antisera to either human PTH or PTHrP.

Without being limited by theory, the results provided in this example indicate that Fugu PTH may have different pharmacokinetics to human PTH. These different pharmacokinetics may mean that Fugu PTH is less likely to cause hypercalcemia, or result in other side-effects, when administered to a human or other animal. Furthermore, the differences between Fugu PTH and human PTH may result in a reduced immune response, such as allergic reactions, to the administered Fugu PTH polypeptide compared to, for example, human PTH.

### EXAMPLE 2

Anabolic Effects of Fugu PTH on Bones of Rats.

Materials and Methods

The anabolic effect of Fugu PTH was assessed in the bones of 50-60 g male Sprague-Dawley rats (3-4 weeks old).

Rats were subcutaneously administered a low or high dose (3 or 10 gg) of Fugu PTH(1-34) per 100 g body weight each day for 30 days. The synthetic PTH peptides were dissolved in 0.01M acetic acid and then daily injections were prepared in normal saline with 2% rat serum (from male Sprague-Dawley rats). The rats were weighed twice a week and the PTH dose adjusted for the increasing weight of each animal.

There were 12 rats in each of the following treatment groups: control, human PTH (low or high dose) and Fugu PTH (low or high dose). Rats were euthanased using asphyxiation and tibiae were removed, leaving most of the muscle on the bone. The samples were placed into freshly prepared 4% paraformaldehyde. They were fixed for 24 hours and then transferred to 70% ethanol in preparation for histomorphometry.

Fixed tibiae were X-rayed and embedded in methylmethacrylate resin as follows: Tibiae were cleaned of muscle, then bisected transversely and trimmed approximately 2 mm on each side using a water cooled slow speed bench saw to provide a flat surface for embedding, parallel to the sagittal midline. Fixed tibiae were dehydrated in acetone by hourly changes of 70% acetone, 90% acetone and 100% acetone (×2). After dehydration, samples were infiltrated with methylmethacrylate resin (85% methylmethacrylate, 15% dibutylphthalate, 0.05% benzoyl peroxide) twice for at least 3 days (minimum total of 6 days). For at least one day of the infiltration procedure, samples were infiltrated under vacuum at room temperature; all other steps were carried out at 4° C. After infiltration, tibiae were embedded in glass scintillation vials on a polymerised methylmethacrylate base in methylmethacrylate resin (85% methylmethacrylate, 15% dibuylphthalate, 3% benzoyl peroxide) in a waterbath in a 37° C. incubator over 48 hours. After polymerisation, a third layer of methacrylate was included (same components as above plus acrylic resin beads) and allowed to polymerise over another 48 hours. After samples were fully polymerised, they were cooled at −20° C. to allow polymer contraction, before the embedded tibiae were released from the glass vials by smashing them with a hammer. Polymerised tibiae were then ground on an electric grinder/ polisher to expose the bone surface, and provide a squared block to sit securely in a microtome. 5 μm sections were cut on a Leica 2165 microtome, spread with 95% ethanol and adhered to glass microscope slides by clamping overnight at 37° C. separated by pieces of bagging plastic. Fully adhered sections were deplasticised in cellosolve for 2×25 minutes, dehydrated in graded ethanols and stained with toluidine blue for standard histomorphometry. Von Kossa staining was carried out to provide composite images.

Histomorphometry was carried out according to standard procedures using the Osteomeasure Image analysis system (Osteometrics, Decatur, Ga.) in the secondary spongiosa, starting 3 mm below the growth plate in a region 3 mm wide by 1.1 mm high. Data was analysed by one-way ANOVA followed by Tukey's post-hoc test to locate significant differences.

Results and Discussion

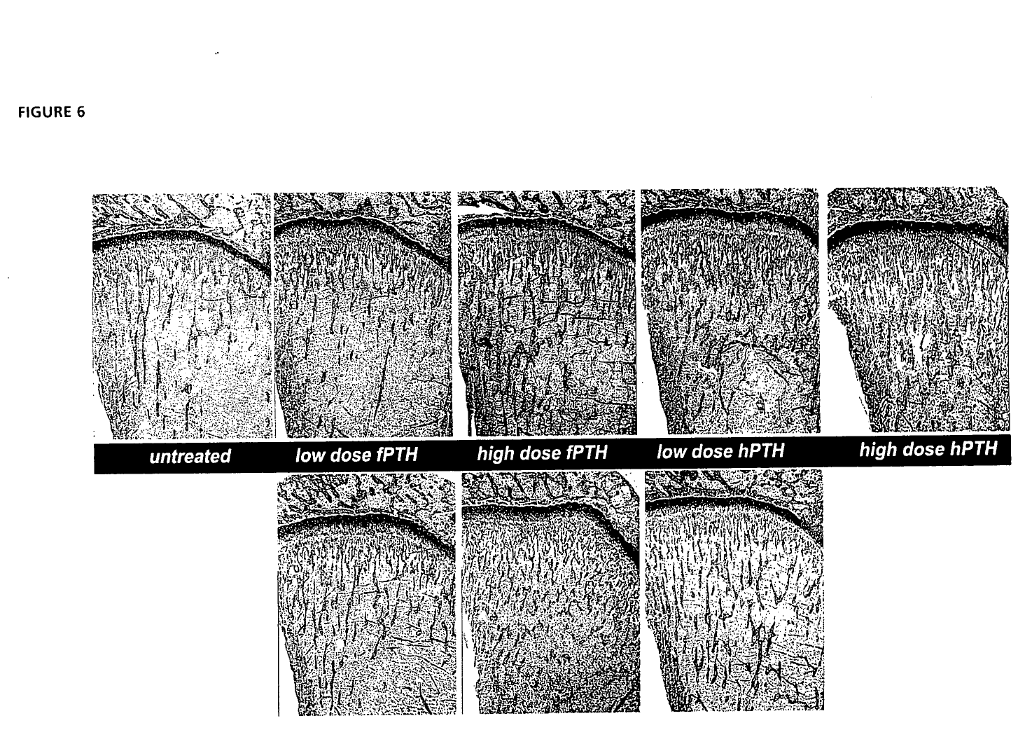

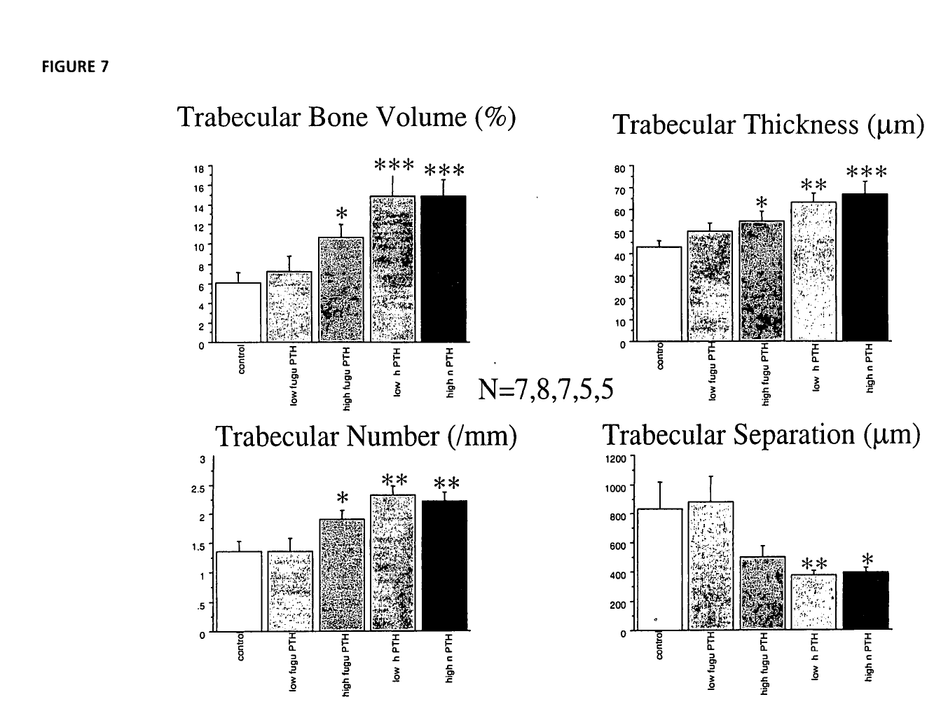

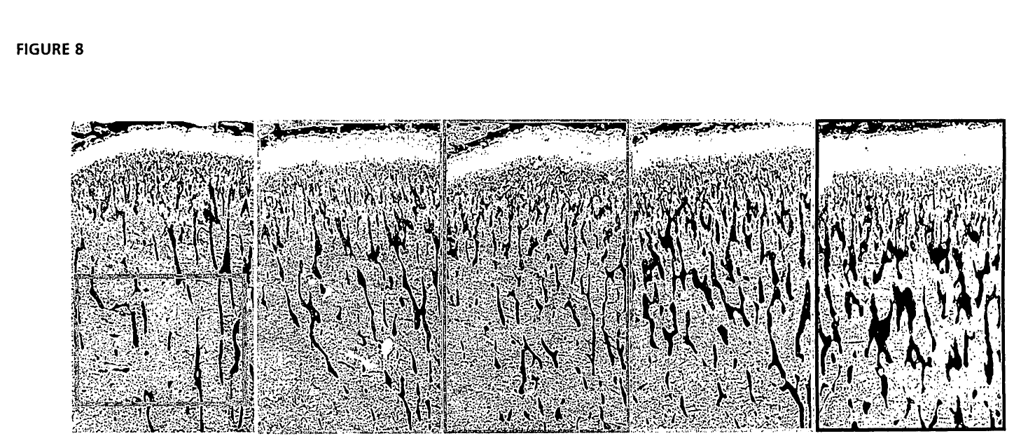

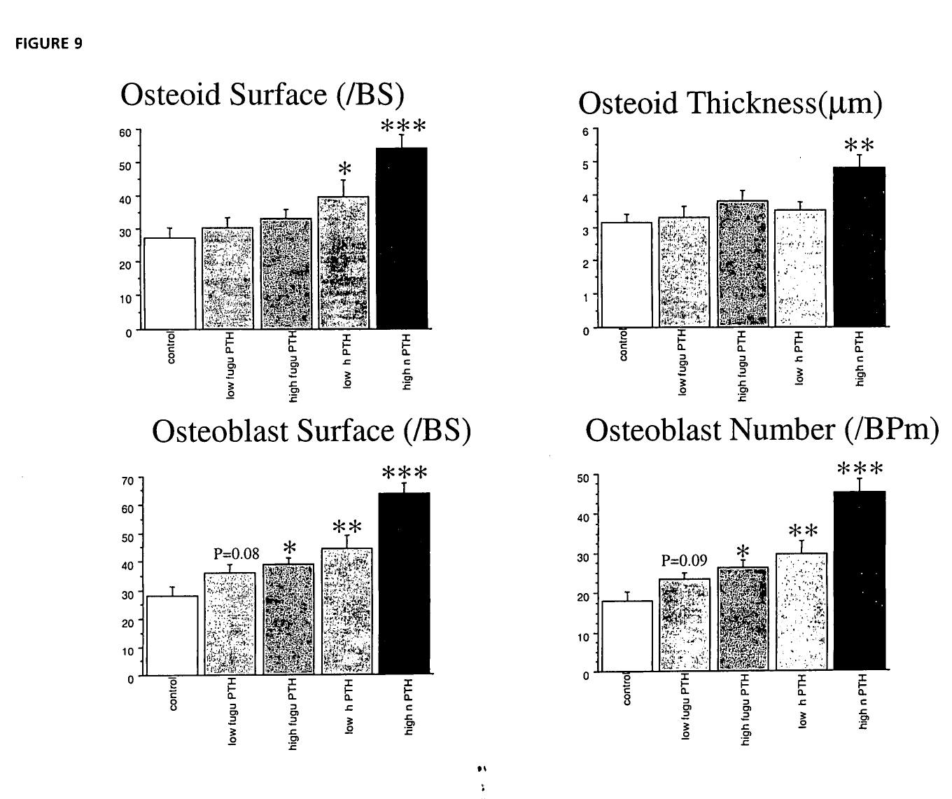

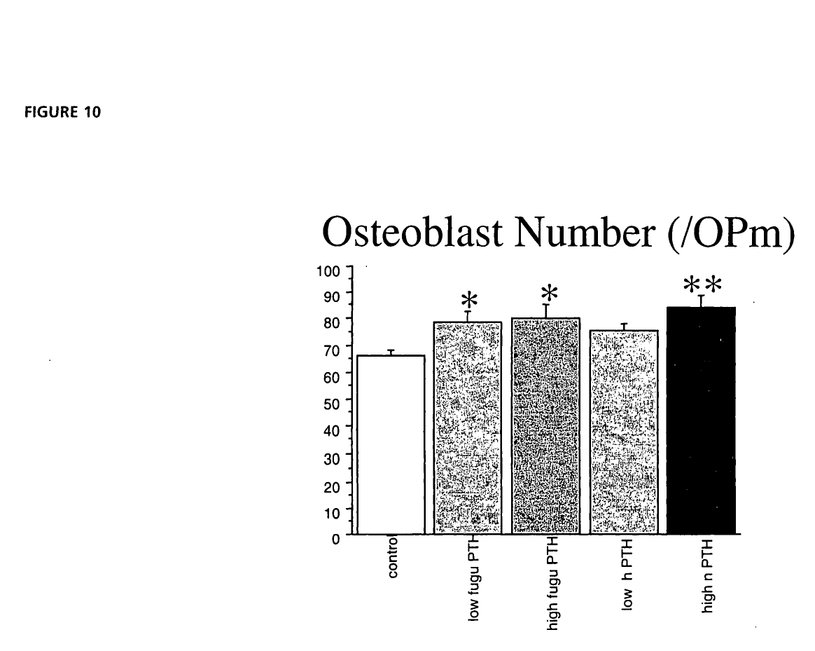

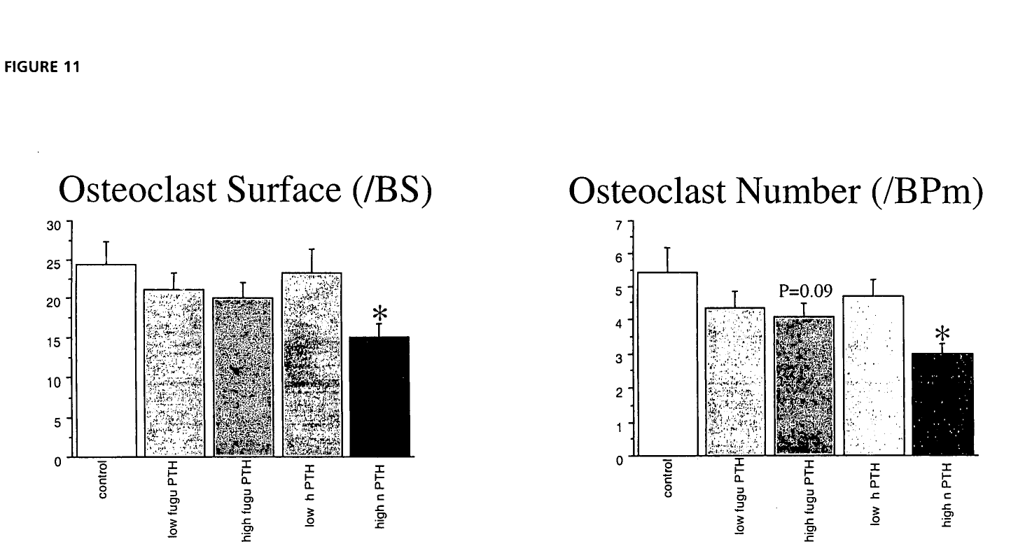

The results obtained in this example are shown in FIGS. 6 to 11. The results show that 10 micrograms/100 grams Fugu PTH(1-34) is anabolic in young growing rats, leading to a significant increase in trabecular bone volume, thickness and trabecular number. This increase in bone mass was associated with increased osteoblast generation, suggesting increased bone formation as the primary mechanism. There was also a trend for reduced osteoclast numbers but this did not reach statistical significance, suggesting that a mild reduction in osteoclastogenesis (observed in the 10 microgram/100 gram human PTH group) may also play a role in the effects of Fugu PTH(1-34) in vivo.

The clinical significance of this finding is that Fugu PTH and biologically active fragments thereof, as well as related polypeptides (eg polypeptides comprisng at least 45% sequence identity to SEQ ID NO: 1), may be of use in the treatment of human osteoporosis and in the prevention of osteoporosis. The effect seen in rats is similar to that observed with human PTH but only on a smaller magnitude. It is therefore expected to have a similar effect on bone in humans to human PTH. The availability of a new polypeptide with similar activity but different amino acid sequence allows for the production of novel PTH analogues which may have more desirable qualities such as improved pharmacokinetics, fewer side effects, different modes of administration or other, as yet, unidentified advantages.

### EXAMPLE 3

Detection of Homologs of fPTH in Other Fish Species.

The aim of this example was to identify previously undescribed genes that encode the parathyroid hormone-like polypeptide in different species of fish.

Methods and Materials

Genomic DNA from muscle samples from the fish species listed in Table 2 was extracted according to standard techniques. Total RNA extracted also as per standard techniques was reverse transcribed using random hexamers to generate cDNA.

Degenerate PCR primers were designed based on amino acid and nucleotide sequences in the N-terminal (highly conserved) and C-terminal (not very well conserved) regions of the Fugu and zebrafish PTH (Table 3). This strategy was not designed to amplify PCR products from genomic DNA which may contain large introns. Thus cDNA was also synthesised to allow detection of PTH genes which may contain large intronic sequences.

In order to detect sequences with low shared homology to Fugu and zebrafish PTH genes, and because degenerate primers were used, an annealing temperature of 45° C. was chosen. The conditions were as follows:

| | | |
|---|---|---|
| 95° C. for 5 mins - initial denaturation | | |
| 95° C. for 1 min - denaturation | | |
| 45° C. for 1 min - annealing | {close oversize brace} | 40 cycles |
| 72° C. for 1 min - extension | | |
| 72° C. for 1 min - final extension | | |

100 nanograms of genomic DNA was used in the PCR reaction using the degenerate primers. PCR products were examined by electrophoresis in a 1.5%-2% agarose gel containing ethidium bromide (EtBr) to allow visualisation of DNA under ultra violet light. Following electrophoresis, PCR products were transferred to a Hybond-N+membrane (Amersham) by the method of Southern transfer. The Southern transfer membranes were hybridised in DIG Easy Hybe (Roche) and probed with a digoxigenin labeled Fugu PTH DNA clone. The conditions for probing used either a hybridisation temperature of 37° C. and washing temperature of 68° C. or a hybridisation temperature of 30° C. and a washing temperature of 37° C. The digoxigenin labeled probe was generated and detected using the DIG High Prime DNA labeling and Detection Starter Kit II (Roche).

TABLE 2

| | Common name | Species name |
|---|---|---|
| F1 | gourami | Trichogaster trichopterus |
| F2 | medaka | Oryzias latipes |
| F3 | cichlid | Metriaclima hajomaylandi |
| F4 | goldfish | Carassius auratus |
| F5 | rainbow shark | Epalzeorhynchus erythrurus |
| F6 | tiger barb | Tertragoza capoeta |
| F7 | Madagascan | Bedotia geayi |
| | rainbowfish | |
| F8 | catfish | Arius graeffei |
| F9 | variatus platy | Xiphophorus variatus |
| F10 | red devil cichlid | Amphilophus labiatum |
| Srgsm1 | gummy shark | Mustelus antarticus |
| Sqgsm2 | gummy shark | Mustelus antarticus |
| | lung fish | Neoceratodus fosteri |
| | Japanese pufferfish | Fugu rubripes |
| | zebrafish | Danio rerio |

TABLE 3

| | | |
|---|---|---|
| Primer | Primer sequence | source |

| | | |
|---|---|---|
| FZPTHfor | 5′-GAAGTWCAAATRCTICAYAA-3′ | degenerate |
| | (SEQ ID NO: 10) | |
| FZPTHrev | 5′-TTIAKIAGTTYTTCIAGNAC-3′ | degenerate |
| | (SEQ ID NO: 11) | |

Wherein, W = A/T, R = A/G, I = inosine (universal base), Y = C/T and N = A/C/G/T.

Results and Discussion

The range of fish species were selected from both bony and cartilaginous fish. Also the species are from both tropical and temperate waters and cover most of the world's geographical areas, including Africa, Asia, South America and Australasia. The Australian lungfish, a lobe-finned fish, constitutes a link between tetrapods and fish which arose during the Devonian period, at least 300 million years ago. The gummy shark is a representative of the cartilaginous fishes, which predate the bony fishes in evolution.

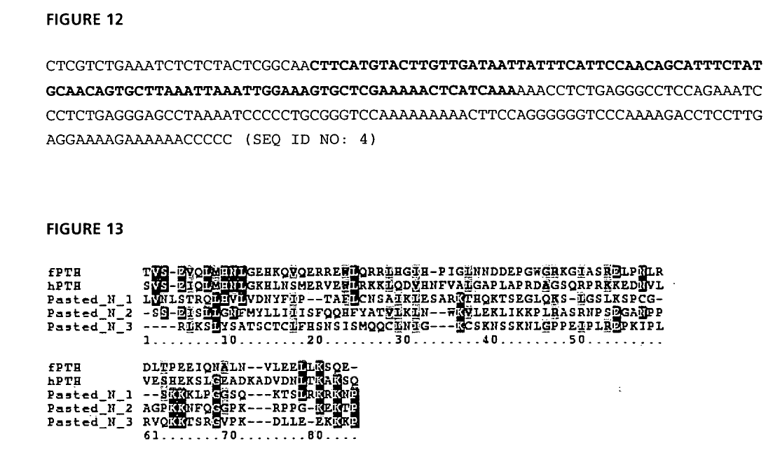

The results are shown in Table 4. Nucleotide sequences homologous to the Fugu PTH DNA, representing likely PTH homologs were detected in gourami, cichlid, goldfish, tiger barb, catfish, red devil cichlid, gummy shark, lung fish and zebrafish. A partial genomic DNA of 236 bp for the likely PTH homolog of gummy shark has subsequently been isolated and sequenced (see FIGS. 12 and 13). The results of the example show a wide distribution of Fugu PTH homologs across fish species. The detection of PTH homologs in cartilaginous fish species was surprising.

The smear observed with the rainbow shark DNA hybridised positively with the digoxigenin labeled Fugu PTH DNA probe. This result suggests that PCR products of varying lengths homologous to the Fugu PTH DNA were synthesised in the reaction, and that further optimisation of the PCR conditions should enable the generation of a discrete band representing a rainbow shark PTH homolog.

TABLE 4

| | gDNA | | cDNA | |
|---|---|---|---|---|
| | | Autoradiography | | Autoradiography |
| PROBE: | EtBr | Fugu | EtBr | Fugu |
| Fish | | | | |
| F1 | neg | neg | 480 bp | neg |
| F2 | neg | neg | neg | neg |
| F3 | neg | neg | 500 bp | neg |
| F4 | 400 bp | pos | neg | neg |
| | 600 bp | neg | | |
| F5 | neg - no | pos/neg | neg | neg |
| | bands | (smear) | | |
| | but | | | |
| | visible | | | |
| | smear | | | |
| F6 | 400 bp | neg | neg | neg |
| | 700 bp | neg | | |
| F7 | neg | neg | neg | neg |
| F8 | 300 bp | neg | neg | neg |
| | 700 bp | neg | | |
| F9 | neg | neg | neg | neg |
| F10 | neg | neg | 350 bp | neg |
| Srgsm1 | 150 bp | neg | neg | neg |
| Sqgsm2 | 150 bp | neg | neg | neg |
| Lung fish | 700 bp | neg | N/A | |
| | 800 bp | neg | | |
| Fugu rubripes | 400 bp | neg | N/A | |
| (expected size = 244 bp) | 500 bp | neg | | |
| | 1200 bp | neg | | |
| zebrafish | 280 bp | neg | N/A | |
| (expected size = 185 bp) | 600 bp | neg | | |

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed anywhere before the priority date of each claim of this application.

It will be appreciated by persons skilled in the art that numerous variations and/or modifications may be made to the invention as shown in the specific embodiments without departing from the spirit or scope of the invention as broadly-described. The present embodiments are, therefore, to be considered in all respects as illustrative and not restrictive.

1. An isolated polypeptide or biologically active fragment thereof, wherein said polypeptide comprises an amino acid sequence with at least 45% sequence identity with SEQ ID NO: 1.

2. The isolated polypeptide or biologically active fragment of claim 1, wherein said polypeptide comprises an amino acid sequence with at least 55% sequence identity with in SEQ ID NO: 1.

3. The isolated polypeptide or biologically active fragment of claim 1, wherein said polypeptide comprises an amino acid sequence with at least 65% sequence identity with SEQ ID NO: 1.

4. The isolated polypeptide or biologically active fragment of claim 1, wherein said polypeptide comprises an amino acid sequence with at least 75% sequence identity with SEQ ID NO: 1.

5. The isolated polypeptide or biologically active fragment of claim 1, wherein said polypeptide comprises an amino acid sequence with at least 90% sequence identity with SEQ ID NO: 1.

6. The isolated polypeptide or biologically active fragment of claim 1, wherein said polypeptide comprises an amino acid sequence with at least 95% sequence identity with SEQ ID NO: 1.

7. The isolated polypeptide or biologically active fragment of claim 1, comprising an amino acid sequence with at least 65% sequence identity to amino acids 1-34 of SEQ ID NO: 1.

8. The isolated polvpeptide or biologically active fragment of claim 1, comprising an amino acid sequence with at least 75% sequence identity to amino acids 1-34 of SEQ ID NO: 1.

9. The isolated polypeptide or biologically active fragment of claim 1, comprising an amino acid sequence with at least 90% sequence identity to amino acids 1-34 of SEQ ID NO: 1.

10. The isolated polypeptide or biologically active fragment of claim 1, comprising an amino acid sequence with at least 95% sequence identity to amino acids 1-34 of SEQ ID NO: 1.

11. The isolated polypeptide or biologically active fragment of claim 1, comprising an amino acid sequence with at least 65% sequence identity at to amino acids 7-34 of SEQ ID NO: 1.

12. The isolated polypeptide or biologically active fragment of claim 1, comprising an amino acid sequence with at least 70% sequence identity to amino acids 7-34 of SEQ ID NO: 1.

13. The isolated polypeptide or biologically active fragment of claim 1, comprising an amino acid sequence with at least 90% sequence identity to amino acids 7-34 of SEQ ID NO: 1.

14. The isolated polvpeptide or biologically active fragment of claim 1, comprising an amino acid sequence with at least 95% sequence identity to amino acids 7-34 of SEQ ID NO: 1.

15. (canceled)

16. The isolated polypeptide or biologically active fragment of claim 1, comprising an N-terminus threonine.

17. The isolated polypeptide or biologically active fragment of claim 7, comprising an N-terminus threonine.

18. A polypeptide or biologically active fragment with parathyroid hormone activity, wherein said polypeptide or fragment comprising an N-terminus threonine.

19. The polypeptide or biologically active fragment of claim 18, wherein the polypeptide or fragment is derived from a bony or cartilaginous fish species.

20. An isolated nucleic acid molecule encoding the polypeptide or biologically active fragment selected from the group consisting of:
a) an amino acid having at least 45% sequence identity to SEQ ID NO: 1,
b) an amino acid having at least 45% sequence identity to SEQ ID NO: 1 and having an N-terminus threonine, and
c) a polypeptide having parathyroid hormone activity and having an N-terminus threonine.

21. An isolated nucleic acid molecule encoding the biologically active fragment selected from the group consisting of:
a) an amino acid sequence having at least 65% sequence identity to amino acids 1-34 of SEQ ID NO: 1, and
b) an amino acid sequence having at least 65% sequence identity to amino acids 1-34 of SEQ ID NO: 1 and having an N-terminus threonine.

22. A nucleic acid molecule comprising the nucleotide sequence of SEQ ID NO: 2.

23. An isolated nucleic acid molecule encoding a polypeptide or biologically active fragment with parathyroid hormone activity, wherein the isolated nucleic acid molecule comprises a nucleotide sequence of SEQ ID NO: 4.

24. A recombinant host, wherein the recombinant host includes a nucleic acid molecule according to claim 20.

25. A pharmaceutical composition comprising a pharmaceutically-acceptable carrier and a biologically active molecule selected from the group consisting of
a) polypeptide comprising an amino acid sequence with at least 45% sequence identity to SEQ ID NO: 1,
b) a polypeptide having at least 45% sequence identity to SEQ ID NO: 1 and having an N-terminus threonine,
c) a polypeptide having parathyroid hormone activity and having an N-terminus threonine
d) a polypeptide having an amino acid sequence with at least 65% sequence identity to amino acids 1-34 of SEQ ID NO: 1, and
e) a polypeptide having an amino acid sequence with at least 65% sequence identity to amino acids 1-34 of SEQ ID NO: 1 and having an N-terminus threonine
f) a nucleic acid comprising the nucleotide sequence of SEQ ID NO: 2
g) a nucleic acid comprising the nucleotide sequence of SEQ ID NO: 4.

26. A method of treating a disease associated with abnormal calcium homeostasis in a subject, the method comprising administering to the subject a therapeutically effective amount of a biologically active molecule selected from the group consisting of:
a) polvpeptide comprising an amino acid sequence with at least 45% sequence identity to SEQ ID NO: 1,
b) a polypeptide having at least 45% sequence identity to SEQ ID NO: 1 and having an N-terminus threonine,
c) a polypeptide having parathyroid hormone activity and having an N-terminus threonine,
d) a polypeptide having an amino acid sequence with at least 65% sequence identity to amino acids 1-34 of SEQ ID NO: 1,
e) a polypeptide having an amino acid sequence with at least 65% sequence identity to amino acids 1-34 of SEQ ID NO: 1 and having an N-terminus threonine,
f) a nucleic acid comprising the nucleotide sequence of SEQ ID NO: 2, and,
g) a nucleic acid comprising the nucleotide sequence of SEQ ID NO: 4.

27. The method of claim 26, wherein the disease is selected from the group consisting of osteoporosis, osteopenia, Paget's disease, bone cancer, hyperparathyroidism, hypoparathyroidism, hypercalcemia, psoriasis and other skin-related conditions.

28. A method of treating a disease that results from altered or excessive action of a PTH receptor, the method comprising administering to a subject a therapeutically effective amount of a biologically active molecule selected from the group consisting of
a) polvpeptide comprising an amino acid sequence with at least 45% sequence identity to SEQ ID NO: 1,
b) a polypeptide having at least 45% sequence identity to SEQ ID NO: 1 and having an N-terminus threonine,
c) a polvpeptide having parathyroid hormone activity and having an N-terminus threonine,
d) a polypeptide having an amino acid sequence with at least 65% sequence identity to amino acids 1-34 of SEQ ID NO: 1,
e) a polvpeptide having an amino acid sequence with at least 65% sequence identity to amino acids 1-34 of SEQ ID NO: 1 and having an N-terminus threonine,
f) a nucleic acid comprising the nucleotide sequence of SEQ ID NO: 2, and,
g) a nucleic acid comprising the nucleotide sequence of SEQ ID NO: 4.

29. A method for determining rates of bone formation, bone resorption and/or bone remodelling comprising:
administering to a subject an effective amount of a labeled biologically active molecule selected from the group consisting of
a) polypeptide comprising an amino acid sequence with at least 45% sequence identity to SEQ ID NO: 1,
b) a polypeptide having at least 45% sequence identity to SEQ ID NO: 1 and having an N-terminus threonine,
c) a polypeptide having parathyroid hormone activity and having an N-terminus threonine,
d) a polypeptide having an amino acid sequence with at least 65% sequence identity to amino acids 1-34 of SEO ID NO: 1,
e) a polypeptide having an amino acid sequence with at least 65% sequence identitv to amino acids 1-34 of SEQ ID NO: 1 and having an N-terminus threonine,
f) a nucleic acid comprising the nucleotide sequence of SEQ ID NO: 2, and,
g) a nucleic acid comprising the nucleotide sequence of SEQ ID NO: 4, and
determining the uptake of said biologically active molecule into the bone of said subject.

30. An antibody, that specifically binds to the a biologically active molecule selected from the group consisting of
a) polypeptide comprising an amino acid sequence with at least 45% sequence identity to SEQ ID NO: 1,
b) a polypeptide having at least 45% sequence identity to SEQ ID NO: 1 and having an N-terminus threonine,
c) a polypeptide having parathyroid hormone activity and having an N-terminus threonine,
d) a polypeptide having an amino acid sequence with at least 65% sequence identity to amino acids 1-34 of SEQ ID NO: 1,
e) a polypeptide having an amino acid sequence with at least 65% sequence identity to amino acids 1-34 of SEQ ID NO: 1 and having an N-terminus threonine,
f) a nucleic acid comprising the nucleotide sequence of SEQ ID NO: 2, and,
g) a nucleic acid comprising the nucleotide sequence of SEO ID NO: 4.

31. The biologically active fragment of claim 1, consisting of amino acids 7-34 of SEQ ID NO: 1.

32. The biologically active fragment of claim 1, consisting of amino acids 1-34 of SEQ ID NO: 1.


SEQUENCE LISTING (14 sequences)

SEQ ID NO: 1 - protein, 80 aa, Fugu rubripes
TVSEVQLMHNLGEHKQVQERREWLQRRLHGIHPIGLNNDDEPGWGRKGIASRELPNLRDLTPEEIQNALNVLEELLKSQE

SEQ ID NO: 2 - DNA, 240 bp, Fugu rubripes
acagtgagtgaagtccagctcatgcacaaccttggggagcataagcaggtgcaggagcgccgtgagtggttacagcgaagactccatggtattcaccctataggtctcaacaacgacgatgaaccaggatggggcagaaaggggatagcttcccgagagctgcctaacctcagagacctgaccccagaagagatccagaatgcattaaatgtcttagaggaactgctcaaatcacaggag

SEQ ID NO: 3 - DNA, 276 bp, Fugu rubripes
acagtgagtgaagtccagctcatgcacaaccttggggagcataagcaggtgcaggagcgccgtgagtggttacagcgaagactccatggtattcaccctataggtctcaacaacgacgatgaaccaggatggggcagaaaggggatagcttcccgagagctgcctaacctcagagacctgaccccagaagagatccagaatgcattaaatgtcttagaggaactgctcaaatcacaggagtgaagattttttttaaatttctttttttattatttg

SEQ ID NO: 4 - DNA, 238 bp, Mustelus antarcticus
ctcgtctgaaatctctctactcggcaacttcatgtacttgttgataattatttcattccaacagcatttctatgcaacagtgcttaaattaaattggaaagtgctcgaaaaactcatcaaaaaacctctgagggcctccagaaatccctctgagggagcctaaaatccccctgcgggtccaaaaaaaaacttccaggggggtcccaaaagacctccttgaggaaaagaaaaaaccccc

SEQ ID NO: 5 - protein, 88 aa, chicken
SVSEMQLMHNLGEHRHTVERQDWLQMKLQDVHSALEDARTQRPRNKEDIVLGEIRNRRLLPEHLRAAVQKKSIDLDKAYMNVLFKTKP

SEQ ID NO: 6 - protein, 84 aa, Homo sapiens
SVSEIQLMHNLGKHLNSMERVEWLRKKLQDVHNFVALGAPLAPRDAGSQRPRKKEDNVLVESHEKSLGEADKADVDNLTKAKSQ

SEQ ID NO: 7 - protein, 127 aa, Fugu rubripes
SVSHAQLMHDKGRSLQEFRRRMWLHKLLEEVHTANEEAPPVQSRTQTQTFSGNSLHEKPPPGATKNLPDRESLDREGTNLPQETNKALAYKDQPLKLATKRKKKARLGRHREADKKRRRARSVAKEP

SEQ ID NO: 8 - protein, 125 aa, sparus
SVSHAQLMHDKGRSLQEFRRRMWLHELLEEVHTADDRPVQSRTQSQTFSGNALHEKPPGATKNIPDRFRLDREGPNLPQETNKALAYKDQPLKVATKRKKKVRLGRRRESDKKRRRARSVTTKEQ

SEQ ID NO: 9 - protein, 111 aa, Homo sapiens
AVSEHQLLHDKGKSIQDLRRRFFLHHLIAEIHTAEIRATSEVSPNSKPAPNTKNHPVRFGSDDEGRYLTQETNKVETYKEQPLKTPGKKKKGKPGKRKEQEKKKRRTRSAW

SEQ ID NO: 10 - DNA, 20 bp, artificial sequence
  oligonucleotide
  misc_feature (6)..(6): W=A or T
  misc_feature (12)..(12): R=A or G
  misc_feature (15)..(15): N=inosine
gaagtwcaaatrctncayaa

SEQ ID NO: 11 - DNA, 20 bp, artificial sequence
  oligonucleotide
  misc_feature (3)..(3): N=inosine
  misc_feature (11)..(11): Y=C or T
  misc_feature (6)..(6): N=inosine
  misc_feature (15)..(15): N=inosine
  misc_feature (18)..(18): N=A or C or G or T
ttnaknagttyttcnagnac

SEQ ID NO: 12 - protein, 76 aa, Mustelus antarcticus
LVNLSTRQLHVLVDNYFIPTAFLCNSAIKLESARKTHQKTSEGLQKSLGSLKSPCGSKKKLPGGSQKTSLRKRKNP

SEQ ID NO: 13 - protein, 77 aa, Mustelus antarcticus
SSEISLLGNFMYLLIIISFQQHFYATVLKLNWKVLEKLIKKPLRASRNPSEGANPPAGPKKNFQGGPKRPPGKEKTP

SEQ ID NO: 14 - protein, 74 aa, Mustelus antarcticus
RLKSLYSATSCTCLFHSNSISMQQCLNIGKCSKNSSKNLGPPEIPLREPKIPLRVQKKTSRGVPKDLLEEKKKP